(19) United States
(12) Patent Application Publication
(10) Pub. No.: US 2011/0104823 A1
(43) Pub. Date: May 5, 2011

(54) MEASURING LIPOPROTEIN CONTAINING PARTICLES

(76) Inventor: Whyte G. Owen, Rochester, MN (US)

(21) Appl. No.: 13/003,621

(22) PCT Filed: Jul. 9, 2009
(86) PCT No.: PCT/US2009/050128

§ 371 Date: Jan. 11, 2011

Related U.S. Application Data
(60) Provisional application No. 61/080,126, filed on Jul. 11, 2008.

(51) Int. Cl. G01N 33/566

(52) U.S. Cl. 436/501

(57) ABSTRACT

This document provides methods and materials involved in assessing samples (e.g., serum samples) for lipoprotein containing particles. For example, methods and materials involved in using anti-apoprotein antibodies (e.g., fluorescently labeled anti-apoprotein antibodies) to label lipoprotein containing particles that can be detected or measured using flow cytometry are provided.

### CROSS-REFERENCE TO RELATED APPLICATIONS

This application claims benefit of priority from U.S. Provisional Application Ser. No. 61/080,126, filed on Jul. 11, 2008.

### BACKGROUND

1. Technical Field

This document relates to methods and materials involved in assessing samples (e.g., serum samples) for lipoprotein containing particles. For example, this document relates to methods and materials involved in using anti-apoprotein antibodies to label lipoprotein containing particles that can be detected or measured using flow cytometry.

2. Background Information

Lipids such as cholesterol bind to apoprotein in serum to form lipoproteins. Lipoproteins are typically classified as chylomicrons, very low density lipoproteins (VLDL), intermediate density lipoproteins (IDL), low density lipoproteins (LDL), and high density lipoproteins (HDL) according to their physical properties. Among them, LDL has been implicated as a substance that induces arteriosclerosis.

### SUMMARY

This document provides methods and materials involved in assessing samples (e.g., serum samples) for lipoprotein containing particles. For example, this document provides methods and materials involved in using anti-apoprotein antibodies (e.g., fluorescently labeled anti-apoprotein antibodies) to label lipoprotein containing particles that can be detected or measured using flow cytometry. In some cases, the methods and materials provided herein can include using flow cytometry to measure the number of chylomicron, VLDL, IDL, LDL, and HDL particles per unit volume of plasma with a single plasma sample. Having the ability to measure the amount of chylomicron, VLDL, IDL, LDL, and HDL particles in a single sample as small as about 5 μL can allow clinicians to identify quickly and accurately patients having an elevated risk of developing arteriosclerosis.

In general, one aspect of this document features a method for assessing a sample for the presence of lipoprotein containing particles. The method comprises, or consists essentially of, (a) contacting the sample from a mammal with a labeled anti-apoprotein antibody under conditions wherein the antibody forms a complex with a lipoprotein containing particle if present within the sample, and (b) using flow cytometry to determine the presence or absence of the complex or the amount of the complex. The mammal can be a human. The lipoprotein containing particle can be LDL. The labeled anti-apoprotein antibody can be an anti-B100 antibody. The method can comprise contacting the sample with multiple differentially labeled anti-apoprotein antibodies. The multiple differentially labeled anti-apoprotein antibodies can comprise a labeled anti-B100 antibody and a labeled anti-apo C antibody. The method can comprise using flow cytometry to determine the amount of differentially labeled complexes, thereby determining the amount of more than one lipoprotein containing particle or combination of lipoprotein containing particles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains plots of flow cytometry data obtained using human serum and anti-B 100 and anti-CD60 antibodies. Five μL of plasma plus 1 mL of buffer was immuno-stained with anti-B-100/48 (FITC) and anti-CD61 (PE). The left pane shows the flow cytometer scattering gates for all events (P1), for microparticles, and for calibrator beads (BD beads). The right pane shows events within the microparticle gate that stained with anti-B100/48 (Q4), which marks chylomicrons +LDL+IDL+VLDL, and with anti-CD61 (Q1), which is a marker for microparticles derived from platelets, which appear as the darker events in the microparticle gate. The analysis reveals that 89% of the microparticle gate are lipoproteins, 5% are from platelets, and 5% are unidentified by either of the two antibodies. Q3: Negative (unstained)—2052 events, 5 percent of total. Q4: B100/48 positive (Lipoproteins)—34482 events, 89 percent of total. Q1: CD61 (platelet microparticles)—1771 events, 5 percent of total. Q2: double positives—372 events, 1 percent of total. Abbreviations: FSC, forward scatter; SSC, side scatter; pe, phytoerythrin; fitc, fluorescein; and BD Beads, internal standard for counting.

### DETAILED DESCRIPTION

This document provides methods and materials involved in assessing samples (e.g., serum samples) for lipoprotein containing particles. For example, this document provides methods and materials involved in using anti-apoprotein antibodies (e.g., fluorescently labeled anti-apoprotein antibodies) to label lipoprotein containing particles that can be detected or measured using flow cytometry. In some cases, the methods and materials provided herein can include using flow cytometry to measure the number of chylomicron, VLDL, IDL, LDL, and HDL particles per unit volume of plasma with a single plasma sample.

As disclosed herein, a blood sample (e.g., a plasma blood sample) from a mammal can be incubated with one or more anti-apoprotein antibodies that are labeled (e.g., fluorescently labeled). The sample can be assessed using flow cytometry to determine the amount of any one or combination of lipoprotein containing particles. Lipoprotein containing particles include, without limitation, chylomicron, VLDL, IDL, LDL, and HDL particles.

Lipoprotein containing particles can be assessed in any mammal including, without limitation, a human, dog, cow, rodent, pig, horse, cat, or goat. Any appropriate sample (e.g., a plasma blood sample) can be used to assess the amount of lipoprotein containing particles present in a mammal, and any appropriate method can be used to obtain a sample from a mammal. For example, a blood sample (e.g., a fasting blood sample) can be obtained from a mammal by peripheral venipuncture. Plasma can be prepared from a blood sample using any standard method. For example, a blood sample can be drawn into a tube containing an anticoagulant (e.g., heparin or sodium citrate). The blood sample can be centrifuged at, for example, about 1,500×g for about 10 minutes. The upper phase can be aliquotted into tubes and can be used immediately or can be stored (e.g., at −70° C.) prior to being used.

Once a sample (e.g., a plasma blood sample) is obtained from a mammal, the sample can be analyzed to determine the amount of any one or combination of lipoprotein containing particles. For example, a portion of a sample can be added to a solution containing an anti-apoprotein antibody labeled with a particular fluorescent label. The solution can contain additional components such as buffers (e.g., HEPES) and Tris. The solution can be incubated for, e.g., 10, 20, or 60 minutes at room temperature. Examples of anti-apoprotein antibodies include, without limitation, apo B-100, apo B-100/48, apo AI, apo AII, apo CII, apo CIII, and apo E antibodies. Typically, when using multiple different anti-apoprotein antibodies in a single reaction to detect a particular lipoprotein containing particle or a particular combination of lipoprotein containing particles, each anti-apoprotein antibody is labeled with a different label. Individual lipoprotein containing particles and combinations of lipoprotein containing particles can be measured using the particular anti-apoprotein antibodies and combinations of anti-apoprotein antibodies as set forth in Table 1. In general, Apo B-48/100 can be present on VLDL, IDL, and LDL, while apo B-48 can be present on chylomicrons, which also can carry some apo E and apo C-II acquired from HDL. Apo E and apo CIII can be present on VLDL particles. Apo A-I, A-II, and CII can be present on HDL particles. HDL also can contain some apo E, which can be transferred to chylomicrons.

TABLE 1
List of anti-apoprotein antibodies for detecting
lipoprotein containing particles.

| Lipoprotein Containing | Anti-Apoprotein |
|---|---|
| Particle | Antibody |
| chylomicron | B48/100 + AI |
| VLDL | B100 + C + E |
| IDL | B100 + E − C |
| LDL | B100 − C-III − E |
| HDL | AI + AII |
| Lp (a) | (a) |

The term “antibody” as used herein refers to intact antibodies as well as antibody fragments that retain some ability to bind an epitope (e.g., an apoprotein epitope). Such fragments include, without limitation, Fab, F(ab')2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics. In some cases, an anti-apoprotein antibody can be an anti-apoprotein monoclonal antibody, an anti-apoprotein polyclonal antibody, a humanized anti-apoprotein antibody, a chimeric anti-apoprotein antibody, or a single chain anti-apoprotein antibody.

Antibodies provided herein can be prepared using any method. For example, any apoprotein, or fragment thereof, can be used as an immunogen to elicit an immune response in an animal such that specific anti-apoprotein antibodies are produced. The preparation of polyclonal antibodies is well-known to those skilled in the art. See, e.g., Green et al., Production of Polyclonal Antisera, in IMMUNOCHEMICAL PROTOCOLS (Manson, ed.), pages 1 5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994; Kohler & Milstein, Nature, 256:495 (1975); and Harlow et al., ANTIBODIES: A LABORATORY MANUAL, page 726 (Cold Spring Harbor Pub. 1988)). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab')2. This fragment can be further cleaved using a thiol reducing agent, and optionally a blocking group for the sulfhydryl groups resulting from cleavage of disulfide linkages, to produce 3.5S Fab' monovalent fragments. In some cases, an enzymatic cleavage using pepsin can be used to produce two monovalent Fab' fragments and an Fc fragment directly. These methods are described, for example, by

Goldenberg (U.S. Pat. Nos. 4,036,945 and 4,331,647). See, also, Nisonhoff et al., Arch. Biochem. Biophys., 89:230 (1960); Porter, Biochem. J., 73:119 (1959); Edelman et al., METHODS IN ENZYMOLOGY, VOL. 1, page 422 (Academic Press 1967); and Coligan et al. at sections 2.8.1-2.8.10 and 2.10.1-2.10.4. In some cases, anti-apoprotein antibodies (e.g., mouse anti-human apoprotein monoclonal antibodies) can be obtained commercially. For example, fluorescently-labeled anti-B48/100 antibodies can be obtained from Abcam (Cambridge, Mass.).

Any appropriate type of label can be attached to an anti-apoprotein antibody to form a labeled anti-apoprotein antibody. For example, a fluorescent label such as fluorescein isothiocyanate (FITC), phycoerythrin (PE), phycoerythrin-Cy5 (PE:Cy5), or allophycocyanin (APC) can be attached to an anti-apoprotein antibody to obtain an fluorescently-labeled anti-apoprotein antibody.

Once a sample has been incubated with a particular anti-apoprotein antibody or combination of anti-apoprotein antibodies, the sample or a portion of the sample can be assessed using flow cytometry. For example, flow cytometry performed using digital flow cytometers such as the BD FACSCanto™ flow cytometer and the Partec ML or SL instruments can be used to determine the level of lipoprotein containing particles within a sample. Standards such as those described herein (e.g., beads of know size) can be used to identify and quantify lipoprotein containing particles present within a sample.

The invention will be further described in the following examples, which do not limit the scope of the invention described in the claims.

### EXAMPLES

### Example 1

### Serum Lipoprotein Measurement Using Flow Cytometry

A blood plasma sample was obtained from a normal human volunteer. About 5 μL of the blood plasma sample were diluted 100 fold with a buffered saline solution. A FITC-labeled goat anti-B48/100 antibody (Abcam; Cambridge, Mass.) and a PE-labeled anti-CD61 antibody (BD Biosciences, San Jose, Calif.) were added to the diluted blood plasma sample to a final concentration of 10 μg/mL. A known amount (50 μL) of fluorescent beads (Trucount™, BD Biosciences; San Jose, Calif.) having an average particle size of 5 to 8 μm was used as an internal standard to calibrate a digital flow cytometer (FACS Canto, BD Biosciences, San Jose, Calif.) for particle size and particle count.

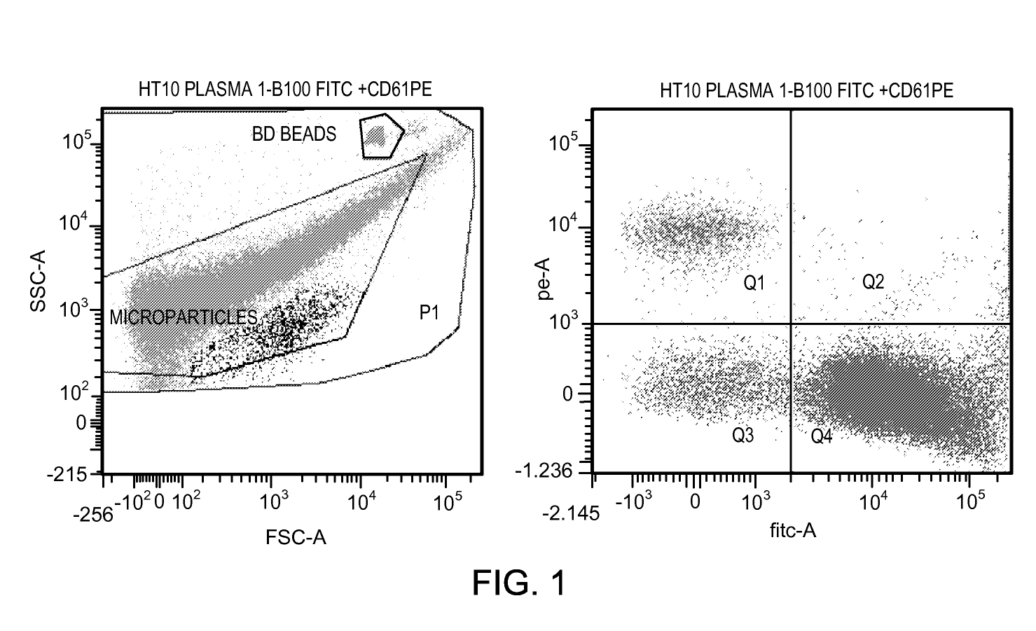

The flow cytometer was gated for the microparticle forward/side scatter cloud (FIG. 1). The samples were then analyzed with the flow cytometer, with output assigned by fluorochrome color. The anti-B48/100 antibody detected microparticles (e.g., chylomicron, VLDL, IDL, and LDL particles), while the anti-CD61 antibody detected microvesicles that can be derived from platelets. These results demonstrate that flow cytometry can be used to detect the number of microparticles present within a serum sample (e.g., an unprocessed serum sample).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a sample for the presence of lipoprotein containing particles, wherein said method comprises:
(a) contacting said sample from a mammal with a labeled anti-apoprotein antibody under conditions wherein said antibody forms a complex with a lipoprotein containing particle if present within said sample, and
(b) using flow cytometry to determine the presence or absence of said complex or the amount of said complex.

2. The method of claim 1, wherein said mammal is a human.

3. The method of claim 1, wherein said lipoprotein containing particle is LDL.

4. The method of claim 1, wherein said labeled anti-apoprotein antibody is an anti-B100 antibody.

5. The method of claim 1, wherein said method comprises contacting said sample with multiple differentially labeled anti-apoprotein antibodies.

6. The method of claim 5, wherein said multiple differentially labeled anti-apoprotein antibodies comprise a labeled anti-B 100 antibody and a labeled anti-apo C antibody.

7. The method of claim 5, wherein said method comprises using flow cytometry to determine the amount of differentially labeled complexes, thereby determining the amount of more than one lipoprotein containing particle or combination of lipoprotein containing particles.
